(19) United States
(12) Patent Application Publication
(10) Pub. No.: US 2014/0054163 A1
(43) Pub. Date: Feb. 27, 2014

(54) METHODS FOR REACTING CYSTEINE RESIDUES IN PEPTIDES AND PROTEINS

(71) Applicant: University of George Risearch Foundation, Inc., Athens, GA (US)

(72) Inventors: VLADIMIR V. POPIK, WATKINSVILLE, GA (US); GEERT-JAN BOONS, ATHENS, GA (US); JUN GUO, ATHENS, GA (US); SELVANATHAN ARUMUGAM, ATHENS, GA (US); EMMANUEL NEKONGO, DULUTH, GA (US); NANNAN LIN, ATHENS, GA (US)

(73) Assignee: University of Georgia Research Foundation, Inc., Athens, GA (US)

(21) Appl. No.: 13/771,378

(22) Filed: Feb. 20, 2013

Related U.S. Application Data
(60) Provisional application No. 61/601,829, filed on Feb. 22, 2012.
(60) Provisional application No. 61/651,082, filed on May 24, 2012.

(51) Int. Cl. C07K 1/13; C12N 9/99

(52) U.S. Cl. CPC C07K 1/13; C12N 9/99
USPC 204/157.68; 530/410; 435/184

(57) ABSTRACT

Methods for labeling or modifying cysteine residues in proteins and/or enzymes are disclosed. The methods include the reaction of an o-naphthoquinone methide with a thiol group of a cysteine residue of a protein or enzyme, which can be reversible in preferred embodiments. The o-naphthoquinone methide can conveniently be generated by irradiation of a precursor compound, preferably in an aqueous solution, suspension, or dispersion.

This application claims the benefit of U.S. Provisional Application Nos. 61/601,829, filed Feb. 22, 2012, and 61/651,082, filed May 24, 2012, both of which are incorporated herein by reference in their entireties.

### GOVERNMENT FUNDING

The present invention was made with government support under Grant No. CHE 0842590, awarded by the National Science Foundation. The Government has certain rights in this invention.

### BACKGROUND

Selective labeling or modification of proteins is an important tool of modern proteomics, medicinal, and biological chemistry. Most commonly, a non-natural amino acid containing a bioorthogonal tag, e.g., an azide, a strained alkene, a terminal acetylene, or the like, is incorporated into the target protein structure. The tag can be incorporated, for example, by metabolic post-synthetic modification of a protein. The specific reaction of the tag is then used to attach a label or immobilize the protein. Cysteine residues, while not bioorthogonal, are also very useful for protein derivatization. Cysteine content of mammalian proteins is about 2% of amino acid residues and most of the cysteines are involved in the formation of intra-protein disulfide bonds. Free, solvent-exposed cysteines are less common in wild-type proteins but are readily introduced into a protein using methods of genetic engineering. The high nucleophilicity of the thiol group allows for the use of Michael addition or nucleophilic substitution reactions for the derivatization of the cysteine side chain. Native chemical ligation protocol also relies on the reaction between an N-terminal cysteine residue and a thioester-containing peptide or protein. A practical approach for derivatization of cysteine residues is the reaction of the cysteine residue with substituted maleamide. Cysteine can be readily converted into dehydroalanine, which can be employed for thiol-ene conjugation.

Additional methods for conveniently labeling or modifying cysteine residues in proteins and/or enzymes are needed in the art.

### SUMMARY

In one aspect, the present disclosure provides a method for labeling a protein having a thiol group. In one embodiment, the method includes: generating an o-naphthoquinone methide; and contacting the o-naphthoquinone methide with the protein under conditions effective to form the labeled protein. In some embodiments, the protein includes a cysteine residue having the thiol group. In some embodiments, the o-naphthoquinone methide is a 2-naphthoquinone-3-methide. In some embodiments, the o-naphthoquinone methide is of the formula:

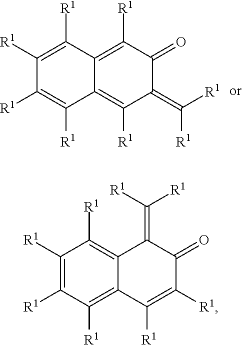

wherein each R¹ is independently H, halogen, or an organic group, wherein two or more R¹ groups may optionally be combined to form one or more rings. Suitable methods for generating an o-naphthoquinone methide are disclosed, for example, in U.S. Patent Application Pub. Nos. 2011/0108411 A1 (Popik et al.) and 2011/0257047 A1 (Popik et al.).

In certain embodiments, the method for labeling the protein having the thiol group is reversible. For such embodiments, the method can further include irradiating the labeled protein under conditions effective to remove the label. Conditions effective to remove the label can include, for example, irradiating the labeled protein in a dilute solution and/or irradiating the labeled protein in the presence of a polarized olefin (e.g., a vinyl ether).

Suitable methods for generating an o-naphthoquinone methide are disclosed, for example, in U.S. Patent Application Pub. Nos. 2011/0108411 A1 (Popik et al.) and 2011/0257047 A1 (Popik et al.). In certain embodiments, generating the o-naphthoquinone methide includes: providing an o-naphthoquinone methide precursor compound; and irradiating the precursor compound under conditions effective to form the o-naphthoquinone methide. In some embodiments, the precursor compound includes a 3-hydroxymethyl-2-naphthol group. In some embodiments, the precursor compound is of the formula:

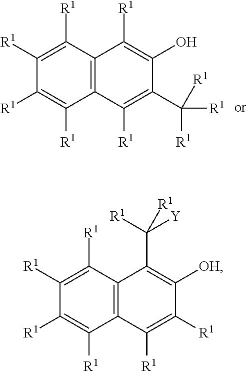

wherein: each R¹ is independently H, halogen, or an organic group, wherein two or more R¹ groups may optionally be combined to form one or more rings; Y is OR⁵, NR⁵₂, or NR⁵₃⁺(Z_{1/q})⁻ wherein Z is an anion having a negative charge of q; and each R⁵ is independently H or an organic group, wherein two or more R⁵ groups may optionally be combined to form one or more rings. Conditions effective to form the labeled protein can include, for example, irradiating a molar excess of the precursor compound compared to the moles of thiol groups of the protein. Optionally, the precursor compound can be irradiated in the presence of the protein.

In another aspect, the present disclosure provides a labeled protein prepared or preparable by any of the methods disclosed herein.

In another aspect, the present disclosure provides a method for inhibiting an enzyme having a thiol group. In one embodiment, the method includes: generating an o-naphthoquinone methide; and contacting the o-naphthoquinone methide with the enzyme under conditions effective to react with the thiol group. In some embodiments, the enzyme includes a cysteine residue having the thiol group. In some embodiments, the enzyme is a cysteine protease. In some embodiments, the o-naphthoquinone methide is a 2-naphthoquinone-3-methide. In some embodiments, the o-naphthoquinone methide is of the formula:

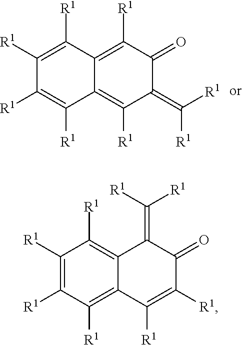

wherein each R¹ is independently H, halogen, or an organic group, wherein two or more R¹ groups may optionally be combined to faun one or more rings.

In certain embodiments, the method for inhibiting the enzyme having the thiol group is reversible. For such embodiments, the method can further include: irradiating the inhibited enzyme under conditions effective to remove the reacted o-naphthoquinone methide and reform the thiol group. Conditions effective to remove the reacted o-naphthoquinone methide fragment and reform the thiol group can include, for example, irradiating the inhibited enzyme in a dilute solution and/or irradiating the inhibited enzyme in the presence of a polarized olefin (e.g., a vinyl ether).

Suitable methods for generating an o-naphthoquinone methide are disclosed, for example, in U.S. Patent Application Pub. Nos. 2011/0108411 A1 (Popik et al.) and 2011/0257047 A1 (Popik et al.). In certain embodiments, generating the o-naphthoquinone methide includes: providing an o-naphthoquinone methide precursor compound; and irradiating the precursor compound under conditions effective to form the o-naphthoquinone methide. In some embodiments, the precursor compound includes a 3-hydroxymethyl-2-naphthol group. In some embodiments, the precursor compound is of the formula:

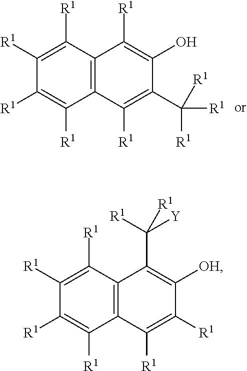

wherein: each R¹ is independently H, halogen, or an organic group, wherein two or more R¹ groups may optionally be combined to form one or more rings; Y is OR⁵, NR⁵₂, or NR⁵₃⁺(Z_{1/q})⁻ wherein Z is an anion having a negative charge of q; and each R⁵ is independently H or an organic group, wherein two or more R⁵ groups may optionally be combined to form one or more rings. Conditions effective to inhibit the enzyme can include, for example, irradiating a molar excess of the precursor compound compared to the moles of thiol groups of the enzyme. Optionally, the precursor compound can be irradiated in the presence of the enzyme.

In another aspect, the present disclosure provides an inhibited enzyme prepared or preparable by any of the methods disclosed herein.

### DEFINITIONS

As used herein, “a,” “an,” “the,” and “at least one” are used interchangeably and mean one or more than one.

As used herein, the term “comprising,” which is synonymous with “including” or “containing,” is inclusive, open-ended, and does not exclude additional unrecited elements or method steps.

As used herein, the term “protein” refers to nitrogenous organic compounds that consist of large molecules composed of one or more long chains of amino acids (e.g., in some embodiments 20 or more amino acids, in other embodiments 50 or more amino acids, and in certain embodiments 100 or more amino acids).

As used herein, the term “enzyme” refers to a protein that has an activity (e.g., catalytic activity) in one or more reactions.

The above brief description of various embodiments of the present invention is not intended to describe each embodiment or every implementation of the present invention. Rather, a more complete understanding of the invention will become apparent and appreciated by reference to the following description and claims in view of the accompanying drawings. Further, it is to be understood that other embodiments may be utilized and structural changes may be made without departing from the scope of the present invention.

### BRIEF DESCRIPTION OF THE DRAWINGS

FIG. 1 is a schematic illustration showing embodiments for photochemical alkylation and release of thiol-containing substrates.

FIG. 2 is a schematic illustration showing embodiments for selective alkylation of cysteine residue in peptides.

FIG. 3 is a schematic illustration showing embodiments for light-driven equilibrium between Naphthoquinone Methide Precursors (NQMP) and NQMP-thioethers (NQMP-SR).

FIG. 4 is a schematic illustration showing embodiments for photochemical uncaging of a peptide cysteine residue.

FIG. 5 is a schematic illustration showing embodiments for selective photochemical alkylation of surface cysteine in bovine serum albumin.

FIG. 6 is an illustration showing Western blot analysis of photochemical labeling (A) and uncaging (B) of bovine serum albumi (BSA). Membranes were stained by antibiotin antibody-HPR-ECL Plus chemiluminescent substrate (for α-biotin) and Coomassie Brilliant Blue dye (for protein).

FIG. 7 is a schematic illustration showing embodiments for biotinylation of BSA using a photochemical Diels-Alder reaction.

FIG. 8 is a schematic illustration showing embodiments for photoglycosylation of bovine serum albumin (BSA).

FIG. 9 is a schematic illustration showing embodiments for photo-triggered catch and release of BSA using Neutravidin Agarose gel.

FIG. 10 is a schematic illustration showing embodiments for photochemical inhibition of cysteine protease activity.

FIG. 11 is a schematic illustration showing embodiments for photochemical re-activation of a protease.

FIG. 12 is a schematic illustration showing embodiments for photochemical re-activation of a protease in the presence of NQMP.

FIG. 13 is a schematic illustration showing the structure of NQMP-triethylene glycol (TEG NQMP-TEG).

FIG. 14 is a graphical illustration showing embodiments for inhibition of papain activity by various concentrations of NQMP-TEG.

FIG. 15 is a graphical illustration showing embodiments for activity of papain vs. different irradiation times.

FIG. 16 is a schematic illustration showing embodiments for preparation of NQMP-D-mannopyranoside conjugate le. Reagents and conditions: (a) i) H₂, Pd/C, MeOH; ii) BrCH₂COCl, Pyridine, DCM; (b) S4, NaOH, DMF; (c) i) 7N NH₃ in MeOH; ii) 2:8 H₂O/AcOH.

FIG. 17 shows an exemplary HPLC analysis of peptides 4, 6, and 7 (0.1 mM in phosphate buffered saline, PBS) photo-labeling with NQMP-TEG (2c, 0.4 mM): Lane 1 shows high pressure liquid chromatography (HPLC) traces after 5 minutes irradiation of with 350 nm light; Lane 2: 2 minutes irradiation at 300 nm; Lane 3: 30 minutes incubation with no irradiation (dark control).

FIG. 18 show exemplary high resolution electrospray ionization mass spectrometry (ESI MS) and tandem mass spectrometry (MS/MS) spectra of peptide-NQMP-TEG conjugate 5.

FIG. 19 shows exemplary UV spectra of PBS solutions of BSA (9a, 10 μM); NQMP-biotin (1d, 10 μM); and BSA-NQMP-Biotin (10a, 7.4 μM).

FIG. 20 is a schematic illustration of the structure of NQMP-Dansyl conjugate (NQMP-DNS).

FIG. 21 is an illustration of an exemplary emission spectrum of Dansyl-derivatized BSA with excitation at 400 nm.

FIG. 22 an illustration of an exemplary PAGE analysis of the photo-conjugation of BSA with NQMP-DNS. Lane 1: MW marker; Lane 3: native BSA; Lanes 4: BSA incubated with NQMP-DNS for 30 min in the dark; Lanes 5: BSA incubated with NQMP-DNS for 90 min in the dark; Lanes 6: Irradiated BSA NQMP-DNS mixture.

### DETAILED DESCRIPTION OF ILLUSTRATIVE EMBODIMENTS

The utility of cysteine-based protein derivatization strategies can be further extended by employing light-induced reactions, as this approach allows for the spatiotemporal control of the process. Several photochemical methods for cysteine derivatization have been reported, including derivatization of proteins using thiol-yne (Conte et al., Chem. Commun. 2011, 47:11086-11088) reaction; removing cross-links in proteins for the studies of folding kinetics (Huang et al., Chem. Commun. 2012, 48:487-497; and Chen et al., Proc. Natl. Acad. Sci. U.S.A. 2004, 101:7305-7310); photochemical inhibition of cysteine protease (Respondek et al., J. Am. Chem. Soc. 2011, 133:17164-17167); and uncaging of cysteine for cross-linking and Au-DNA conjugation (Takada et al., Tetrahedron Lett., 2012, 53(1):78-81).

While many of these techniques work well in some applications, they suffer from common drawbacks. Reactions of electrophiles with free cysteines often produce non-specific side reactions. In aqueous solutions, a huge excess of expensive labeling reagents may be needed in the presence of a nucleophilic solvent.

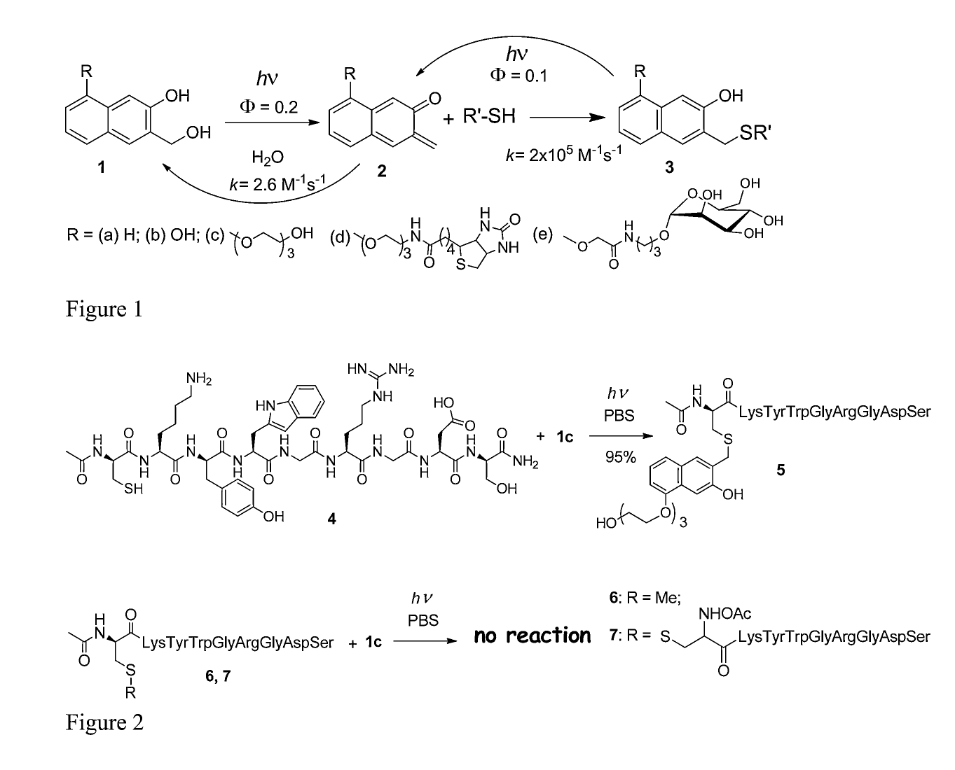

The efficient generation of 2-napthoquinone-3-methides (oNQMs, 2) by photochemical dehydration (Φ=0.20) of 3-(hydroxymethyl)-2-naphthols (NaphthoQuinone Methide Precursors, NQMP 1, FIG. 1) has recently been reported. The lifetime of oNQM (2) in neutral aqueous solutions is only 7 ms as it rapidly adds water (k_{H2O} is approximately 2.6 M⁻¹ seconds⁻¹) to regenerate starting NQMP (1) (Arumugam et al., J. Am. Chem. Soc. 2009, 131:11892). Among endogenous nucleophiles only thiols are reactive enough (k_{RSH} is approximately 2.2×10⁵ M⁻¹ seconds⁻¹) to out-compete water in Michael addition to oNQM (Arumugam et al., J. Am. Chem. Soc. 2009, 131:11892). The thioether (3) produced in the reaction of thiols with oNQM is hydrolytically stable but can be quantitatively cleaved under 300 or 350 nm irradiation back to 2 with 10% quantum yield (FIG. 1) (Arumugam et al., J. Am. Chem. Soc. 2011, 133:5573). The present specification discloses a new method for reversible photochemical derivatization of proteins and peptides, which is based on selective reaction of oNQMs 2 with solvent-exposed cysteine residues. In this approach, irradiation of an aqueous solution of a protein or a peptide containing 4-fold excess of NQMP 1 results in the selective and quantitative labeling of free cysteine residues. Photolysis of NQMP-caged substrates in the absence of labeling reagent can restore free cysteines. The utility of this photo-derivatization—uncaging strategy for pulling down and releasing proteins is also demonstrated herein.

### Precursor Compounds.

In one embodiment, an o-naphthoquinone methide is generated from a precursor compound having one of the formulas:

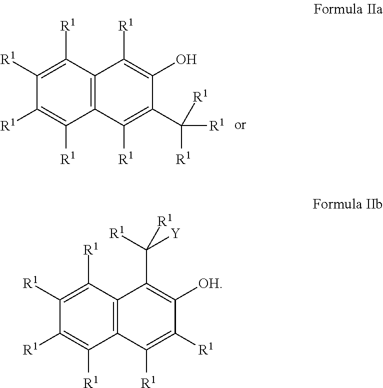

wherein: each R¹ is independently H, halogen, or an organic group; Y is OR⁵, NR⁵₂, or NR⁵₃⁺(Z_{1/q})⁻ wherein Z is an anion having a negative charge of q; and each R⁴ and R⁵ is independently H or an organic group. Optionally, two or more R¹ groups may be combined to form one or more rings (e.g. to form a naphthalene ring or an anthracene ring). Optionally, two or more R⁵ groups may be combined to form one or more rings.

When R¹ and/or R⁵ represent an organic group, preferably the organic group is a carbon-bound (i.e., the bond to the group is to a carbon atom of the organic group) organic group. In certain embodiments, the organic group is an aliphatic group such as a C1-C20 aliphatic group, in some embodiments a C1-C10 aliphatic group, and in some embodiments a C1-C10 hydrocarbon moiety.

As used herein, the term “organic group” is used for the purpose of this disclosure to mean a hydrocarbon group that is classified as an aliphatic group, cyclic group, or combination of aliphatic and cyclic groups (e.g., alkaryl and aralkyl groups). In the context of the present disclosure, suitable organic groups for o-naphthoquinones or precursors thereof, as described herein, are those that do not interfere with a light-induced photodehydration reaction and/or alkylation-dealkylation reactions with a thiol group. In the context of the present disclosure, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

In some embodiments, a detectable label can be attached to the precursor compound and/or the o-naphthoquinone methide. As used herein, a detectable label is meant to include any group or functionality desired that can be detected before and/or after attachment to the surface of a substrate. The labels can be detected by a wide variety of convenient methods including, but not limited to, fluorescence, phosphorescence, radiation detection, optical and electrochemical methods, surface plasmon resonance imaging (SPRi), or combinations thereof. In some embodiments, the detectable label can include a probe molecule. A wide variety of probe molecules can be used including, for example, DNA, peptides, polypeptides, proteins, and combinations thereof.

Generation of o-Naphthoquinone Methide.

Precursor compounds as disclosed herein can be irradiated to generate o-naphthoquinone methides. Typically, the precursor compound is irradiated in an aqueous solution, suspension, or dispersion. As used herein, an aqueous solution, suspension, or dispersion is intended to include liquids that include, but are not limited to, water. Thus, aqueous liquids can also include, for example, organic solvents such as acetonitrile.

Typically, the aqueous solution, suspension, or dispersion of the precursor compound is irradiated at a wavelength of 250 nm to 350 nm. Convenient wavelengths include, for example, 350 nm such as those available from a fluorescent UV lamp. Other convenient wavelengths include, for example, 266 nm and 355 nm. Typically, the aqueous solution, suspension, or dispersion of the precursor compound is irradiated under ambient conditions for a time sufficient for the desired reactions to occur. It would be clear to one of skill in the art that suitable irradiation times can be varied depending on a number of factors such as intensity of the irradiation and the area or volume being irradiated. An exemplary suitable time for the irradiation can be 0.5 minutes to 5 minutes.

o-Naphthoquinone methide.

Irradiation of the precursor compound can generate an o-naphthoquinone methide having one of the formulas:

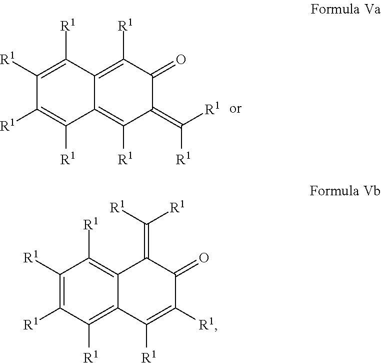

wherein each R¹ is independently H, halogen, or an organic group. Optionally, two or more R¹ groups may be combined to form one or more rings (e.g. to form an o-anthraquinone methide).

When R¹ represents an organic group, preferably the organic group is a carbon-bound (i.e., the bond to the group is to a carbon atom of the organic group) organic group. In certain embodiments, the organic group is an aliphatic group such as a C1-C20 aliphatic group, in some embodiments a C1-C10 aliphatic group, and in some embodiments a C1-C10 hydrocarbon moiety.

### Reversibility

In certain embodiments, the method for labeling the protein having the thiol group is reversible. For such embodiments, the method can further include irradiating the labeled protein under conditions effective to remove the label. Conditions effective to remove the label can include, for example, irradiating the labeled protein in a dilute solution and/or irradiating the labeled protein in the presence of a polarized olefin (e.g., a vinyl ether).

In certain embodiments, the method for inhibiting the enzyme having the thiol group is reversible. For such embodiments, the method can further include: irradiating the inhibited enzyme under conditions effective to remove the reacted o-naphthoquinone methide and reform the thiol group. Conditions effective to remove the reacted o-naphthoquinone methide fragment and reform the thiol group can include, for example, irradiating the inhibited enzyme in a dilute solution and/or irradiating the inhibited enzyme in the presence of a polarized olefin (e.g., a vinyl ether).

Conveniently, irradiation of the labeled protein and/or the inhibited enzyme can regenerate an o-naphthoquinone methide, which can react with a polarized olefin in a hetero-Diels-Alder reaction.

Suitable polarized olefins include, for example, those of the formula:

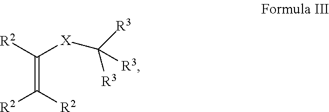

wherein each R² is independently H or an organic group; each R³ is independently H, halogen, or an organic group; and X is O or NR⁴, wherein R⁴ is H or an organic group. Certain polarized olefins include vinyl ethers (X is O).

When R², R³, or R⁴ represents an organic group, preferably the organic group is a carbon-bound (i.e., the bond to the group is to a carbon atom of the organic group) organic group. In certain embodiments, the organic group is an aliphatic group such as a C1-C20 aliphatic group, in some embodiments a C1-C10 aliphatic group, and in some embodiments a C1-C10 hydrocarbon moiety.

The following examples are offered to further illustrate various specific embodiments and techniques of the present disclosure. It should be understood, however, that many variations and modifications understood by those of ordinary skill in the art may be made while remaining within the scope of the present disclosure. Therefore, the scope of the disclosure is not intended to be limited by the following examples.

### Example 1

### Selective and Reversible Photochemical Derivatization of Cysteine Residues in Peptides and Proteins

A method for the selective derivatization of solvent-exposed cysteine residues in peptides and proteins is reported herein. Irradiation of an aqueous solution containing 3-(hydroxymethyl)-2-naphthol derivatives (NQMPs) and protein or peptide with a hand-held 350 nm fluorescent lamp results in fast and efficient conversion of free cysteines into NQMP-thioethers, while other amino acid residues remain unchanged. 0.4 mM of NQMP and 5 minutes of exposure is sufficient for quantitative derivatization of the substrate. NQMP can be equipped with various groups (NQMP-TAGs), such as PEG, dyes, carbohydrates, or possess a fragment for a sandwich assay, e.g., biotin. NQMP-TAG solution employed for the derivatization can be re-used numerous times. The NQMP derivatization is reversible and the native substrate can be released by irradiation in a dilute solution in the absence of the NQMP-TAG or in the presence of ethyl vinyl ether. Reversible biotinylation of bovine serum albumin, as well as quantitative capture and release of this protein using NeutrAvidin Agarose resin beads have been demonstrated herein.

This procedure allows for quantitative capture and for traceless release of protein in a high yield. The cleavage of protein from the solid support does not require any reagents, simplifying purification. Reversible modification of the cysteine residues in proteins can be employed for the photoregulation of protein activity either by caging of catalytically active cysteine (e.g., in cysteine proteases) or by attaching activity-modifying fragment to it. Reversible PEGylation of active peptides and proteins can be employed for light-directed drug delivery or time-resolved activation of these substances.

### Results and Discussion

The 3-hydroxy-2-naphthalenemethanol chromophore (1a) has two major absorption bands in UV region above 210 nm: at λₘₐₓ=275 nm (log ε=4.06) and at λₘₐₓ=324 nm (log ε=3.70). Introduction of an alkoxy substituent at the 5-position of the chromophore (e.g., 1c, FIG. 1; Arumugam et al., J. Am. Chem. Soc. 2011, 133:5573) results in an approximate 10 nm bathochromic shift of both bands. The longer wavelength absorbance band extends past 360 nm making this chromophore suitable for activation using 350 nm fluorescent lamp or 355 nm emission from frequency tripled Nd:YAG laser. o-Naphthoquinone methide precursors 1a-d also show significant fluorescence with a short lifetime (Φ_{Fl}=0.23±0.002 with τ_{FL} is approximately 7 nanaseconds for 1a; (Arumugam et al., J. Am. Chem. Soc. 2009, 131:11892). The emission spectrum of 1a in aqueous solutions contains two major bands at 360 nm and 423 nm. While the parent NQMP 1a and 6-(hydroxymethyl)naphthalene-1,7-diol 1b (Arumugam et al., J. Am. Chem. Soc. 2011, 133:5573) have rather low aqueous solubility, triethylene glycol (NQMP-TEG, 1c), biotin (NQMP-Biotin, 1d; Arumugam et al., J. Am. Chem. Soc. 2011, 133:15730), and mannose (NQMP-Mann, 1e) derivatives are freely soluble in wholly aqueous solutions up to millimolar concentrations (FIG. 1).

Irradiation of NQMP at 300 or 350 nm 1a-e results in dehydration and the formation of o-naphthoquinone methides (oNQM 2a-e). The latter species, as o-quinone methide in general (Quinone Methides. Wiley Series of Reactive Intermediates in Chemistry and Biology, Volume 1, Edited by S. E. Rokita; Wiley: Hoboken, N.J.; 2009; Leo et al., J. Org. Chem. 2003, 68:9643; Chiang et al., J. Am. Chem. Soc. 2000, 122:9854; Diao et al., J. Am. Chem. Soc. 1995, 117:5369; Wang et al., J. Org. Chem. 2005, 70:4910), are very good Michael acceptors because nucleophilic attack on the methide carbon restores the aromaticity of the system. However, in neutral aqueous solutions only very reactive nucleophiles, such as azide ion or thiols, can out-compete hydration back to starting NQMP 1 (Arumugam et al., J. Am. Chem. Soc. 2009, 131:11892). To test the selectivity of oNQM 2c addition to various nucleophilic groups present in proteins, the peptide N-AcSerLysTyrTrpGlyArgGlyAspSer (4, FIG. 2) has been prepared. This nonapeptide contains amino acid residues with nucleophilc side chains: thiol (cysteine), primary amine (lysine), phenol (tyrosine), indole (tryptophan), guanidine (arginine), carboxylate ion (aspartate), and primary alcohol (serine).

Irradiation of PBS solution of peptide 4 containing 4 equivalents of NQMP 1c with hand-held fluorescent 4W UV lamp results in quantitative conversion of starting peptide into the mono-NQMP-TEG labeled derivative 5 (FIG. 2, Table 1). Peptide 5 was isolated by preparative HPLC in 95% yield and its structure confirmed by ESI-HRMS and MS/MS experiment.

To test the cysteine specificity of NQMP photo-click reaction, two analogs of peptide 4 containing the same amino acid sequence but lacking free cysteine were prepared. In nonapeptide 6 cysteine residue was replaced with methionine, while the thiol group in 4 was converted into disulfide bond by oxidative dimerization to form oligopeptide 7. Irradiation of peptides 6 and 7 in the presence of 4 to 12 equivalents of NQMP-TEG (1c) produced no labeled products. HPLC analysis of photolysate showed only presence of starting peptides and 1c. These experiments show the high specificity of addition of photochemically generated oNQM to cysteine thiol group.

It is important to note that peptide 4 and NQMP-TEG (1c) are photochemically stable under these conditions. No decomposition or losses of materials were detected by HPLC after separate irradiation of 0.1 mM PBS solutions of 1c and peptide 4 for 12 minutes using both 300 and 350 nm fluorescent lamps. Obviously, NQMP 1c still forms oNQM 2c but in the absence of thiols the latter rapidly undergoes hydration back to starting materials.

Achieving complete conversion of 0.1 mM solution of 4 to 5 requires 2 minutes of irradiation at 300 nm. Photolysis using 350 nm lamp takes somewhat longer (Table 1), apparently due to the lower extinctions coefficients of NQMP 2c at this wavelength (Arumugam et al., J. Am. Chem. Soc. 2011, 133:5573).

TABLE 1
Photo-labeling of peptide 4 with o-NQMP 1c^{a)}

| Irradiation | Irradiation | | Yield of | |
|---|---|---|---|---|
| wavelength | time | 4 to 1c | conjugate 5 | Unreacted 4 |
| (nm) | (minutes) | ratio | (%) | (%) |
| 300 | 2 | 1:4 | 94 ± 2 | n.d.^{b)} |
| 350 | 5 | 1:4 | 95 ± 2 | n.d.^{b)} |
| 300 | 2 | 1:3 | 79 ± 3 | 17 ± 2 |
| 300 | 2 | 1:2 | 47 ± 2 | 50 ± 2 |
| 300 | 2 | 1:1 | 20 ± 2 | 76 ± 2 |
| 300 | 12 | 1:1 | 18 ± 2 | 79 ± 2 |

^{a)}Starting concentration of peptide 4 = 0.1 mM in PBS at room temperature;
^{b)}Not detected.

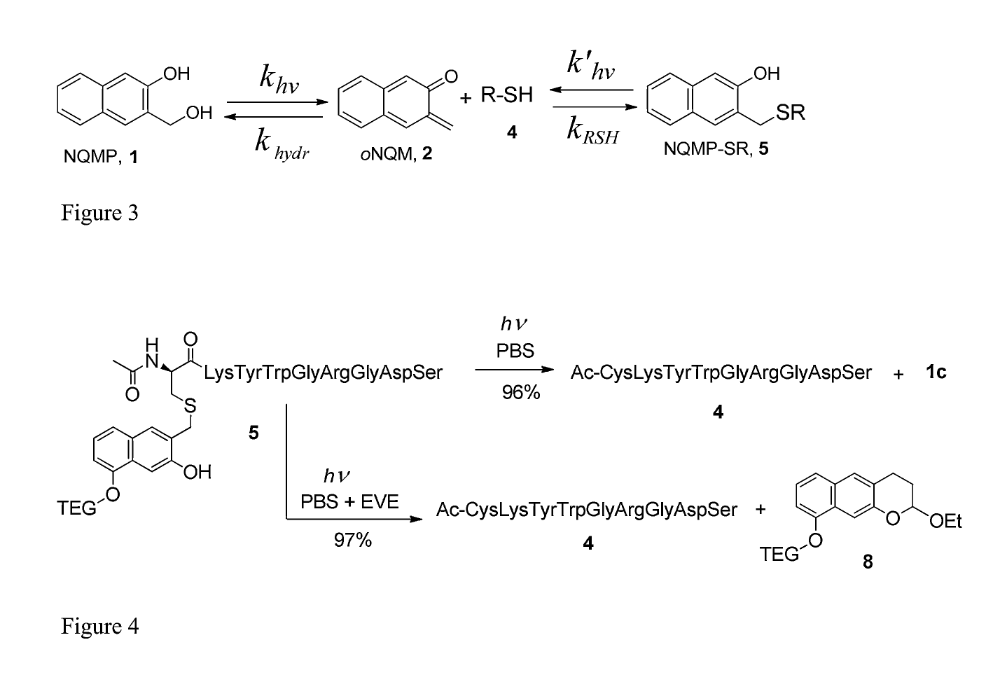

The NQMP-TEG-peptide 5 is photoreactive itself and produces oNQM 2 upon irradiation. The formation of 5, therefore, represents a photochemically driven equilibrium between peptide 4 and NQMP 1c one side and 5 on the other (FIG. 3).

The position of equilibrium is defined by the rate of reaction of the common intermediate 2 with water (k_{hydr}=k_{H2O}[H₂O]) and cysteine (k_{RSH}), as well as by the efficiency of the photoelimination reaction of NQMP and NQMP-SR (kₕᵥ and k′ₕᵥ, respectively). Since under the established equilibrium conditions

$$\frac{d [ NQMP ]}{d t} = \frac{d [ NQMP \text{-} SR ]}{d t} = 0 ,$$

the following equation (eq. 1) can be written, where [NQMP], [RSH], and [NQMP-SR] are equilibrium concentrations of corresponding substrates:

$$\begin{matrix}\frac{{k}_{hv} [ NQMP ]}{{k}_{hydr}} = \frac{{k}_{hv}^{′} [ NQMP \text{-} SR ]}{{k}_{RSH} [ RSH ]} & ( 1 ) \\ \frac{[ NQMP \text{-} SR ]}{[ RSH ]} = \frac{{k}_{hv} {k}_{RSH} [ NQMP ]}{{k}_{hv}^{′} {k}_{hydr}} & ( 2 )\end{matrix}$$

The ratio of caged to free thiol can be then expressed as a function of NQMP concentration (eq. 2). Since oNQMs 2 reacts about five orders of magnitude faster with thiols (k_{RSH} is approximately 2.2×10⁵ M⁻¹ second⁻¹) than with water (k_{H2O} is approximately 2.6 M⁻¹ second⁻¹) and thioethers (e.g., 3 or 5) are 50% less prone to photoelimination than NQMP. NQMP-TEG—peptide adduct is formed in aqueous solutions despite huge excess of nucleophilic solvent (Table 1). At 0.1 mM concentration of peptide 4 almost quantitative conversion is achieved with 4 equivalents of 1c. At lower NQMP to peptide ratios the conversion is reduced, and at equimolar concentrations of both components yield of labeled peptide 5 drops to 20%. These results agree well with the conversion predicted by the equation 2. Prolonged irradiation has virtually no effect on peptide 4 to 5 ratio suggesting that system reaches photostationary state after 2 minutes of irradiation with 300 nm light (Table 1). Equation 2 permits evaluation of the NQMP concentration required to achieve high yield in thiol conjugation experiments. Using experimental rates of oNQM hydration and reactions with simple thiols (Arumugam et al., J. Am. Chem. Soc. 2009, 131:11892), as well as photochemical efficiencies of NQMP and NQMP-SR reactions, it can be determined that 90+% conversion of a substrate is achieved at starting NQMP concentration equal [NQMP]=3×10⁻⁴ M⁻¹+[Substrate]. In biochemical labeling experiments, where concentration of a substrate is in micromolar range or lower, 0.4 mM of NQMP derivative should be sufficient to achieve complete derivatization.

Equation 2 also suggests that at low concentration of free NQMP, the process can be reversed to achieve photochemical release of cysteine residues from NQMP thioether. To explore the efficiency of this uncaging reaction various concentration of purified NQMP-TEG—conjugated peptide (5) were irradiated in aqueous PBS solutions (FIG. 4, Table 2).

Irradiation of 0.1 mM solution of 5 for 2 minutes with 300 nm resulted in the formation of the mixture containing 75% the free peptide 4 and 21% of caged 5. Longer irradiation does not significantly affect the 4 to 5 ratio (Table 2). Very similar product ratio was obtained in the photolysis of 0.1 mM peptide 4 solution in the presence of equimolar amount of NQMP-TEG (1c) (Table 1). These observations indicate that the system photo-equilibrates to same photo-stationary state starting either from NQMP+peptide 4 or from caged peptide 5 side. As expected from the equation 2, further dilution of the solution of 5 results in higher yields of release of the thiol group. At 20 μM concentration or below concentration of the remaining caged substrate 5 fell below HPLC detection limit (Table 2). For practical applications, it can be determined that 90+% release of NQMP-caged thiol is achieved at of 40 μM or lower substrate concentration.

Photorelease of thiol groups can be achieved at any concentration of a substrate when vinyl ether derivatives are employed as oNQM trapping agents. Very facile Diels-Alder addition of ethyl vinyl ether to intermediate oNQM converts the latter into a photostable benzochroman 8 (Arumugam et al., J. Am. Chem. Soc. 2009, 131:11892; and Arumugam et al., J. Am. Chem. Soc. 2011, 133:5573). This reaction efficiently removes oNQM precursors from the reaction mixture and shifts photo-equilibrium towards substrates with free thiol. Due to irreversibility of Diels-Alder addition, even one equivalent ethyl vinyl ether is sufficient to achieve complete conversion of NQMP-caged peptide 5. The cleavage of NQMP-SR thioethers with or without vinyl ether derivatives can be accomplished using 350 nm light. Complete conversion of the starting material under these conditions requires 2-3 times longer irradiation than with 300 nm lamp (Table 2).

TABLE 2
Uncaging of o-NQMP labeled peptide 5^{a)}

| Irradiation | Concentration | Concentration | Unreacted | Yield of |
|---|---|---|---|---|
| time (min) | of 5 (μM) | of EVE (μM) | 5 (%) | peptide 4 (%) |
| 2 | 100 | 0 | 21 ± 2 | 75 ± 3 |
| 12 | 100 | 0 | 19 ± 2 | 77 ± 3 |
| 2 | 50 | 0 | 4 ± 1 | 92 ± 2 |
| 2 | 20 | 0 | n.d.^{b)} | 96 ± 2 |
| 5 | 20 | 0 | n.d.^{b)} | 94 ± 2^{c)} |
| 2 | 100 | 100 | n.d.^{b)} | 97 ± 2 |
| 5 | 100 | 100 | n.d.^{b)} | 96 ± 2^{c)} |

^{a)}Using 300 nm fluorescent lamps in PBS solution at room temperature;
^{b)}Not detected.
^{c)}350 nm fluorescent lamps were used.

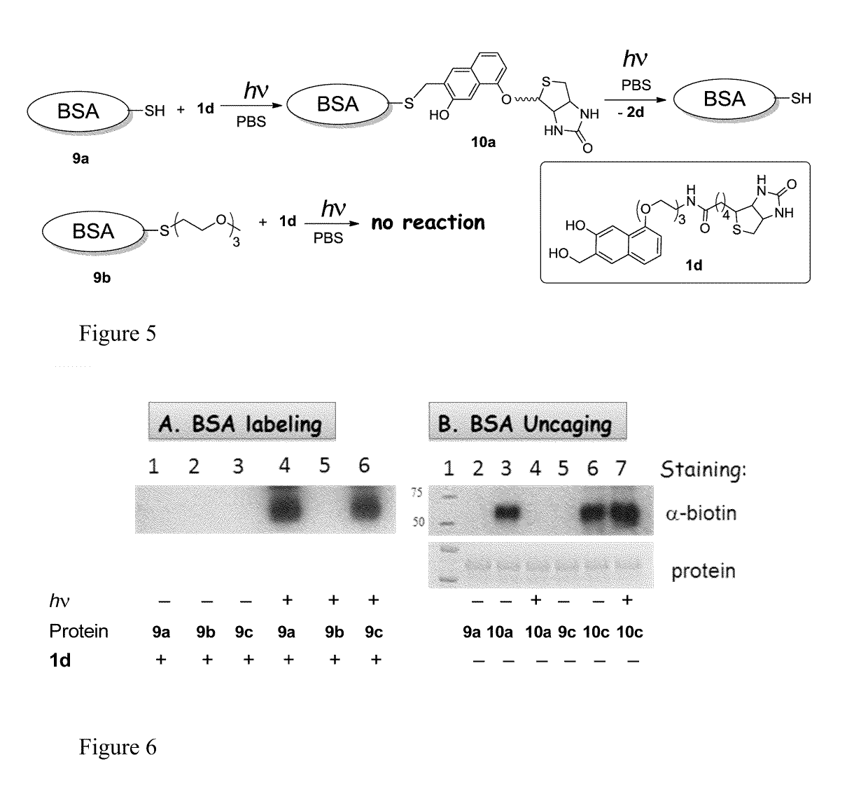

Successful experiments with peptide labeling prompted us to explore the feasibility of the selective caging of free cysteine residues in proteins. The photoclick chemistry was tested on the example of bovine serum albumin (BSA, 9a), which contains the solvent-exposed cysteine (Cys-34), the single free cysteine in this protein (Rondeau et al., Arch. Biochem. Biophys. 2007, 460:141-150). BSA analog 9b, in which Cys-34 was converted into TEG ether, was used as control. PBS solutions containing 11 μm of protein (9a or 9b) and 100 μm of NQMP-biotin (1d; Arumugam et al., J. Am. Chem. Soc. 2011, 133:15730) were irradiated with 350 nm fluorescent lamp for 2 minutes (FIG. 5). Proteins from these experiments, as well as samples of BSA (9a) and BSA-TEG (9b) incubated with 100 μm of 1d in the dark, were isolated by spin filtration and analyzed using Western Blot method. The membrane was stained using with antibiotin antibody—HRP conjugate (FIG. 6). Only native BSA (9a) underwent photo-derivatization to form BSA-NQMP-biotin conjugate 10a (FIG. 6A, lane 4), no biotinylation was observed in the dark or with cysteine-blocked protein 9b.

To evaluate the efficiency of derivatization, total amount of protein and total amount of biotin in the sample of 10a have been determined (Table 3). Total protein was measured using spectrophotometric method and Bradford assay. Results from both methods show very good agreement (Table 3). Biotin was quantified using avidin fluorescence enhancement assay. The concentration of NQMP chromophore in the solution of 10a has also been determined spectroscopically. Results from spectroscopic measurements and quantification assays confirmed the quantitative biotinylation of BSA (Table 3). It is interesting to note that biotinylation of the protein requires lower concentrations of NQMP to achieve full conversion than NQMP concentration needed for the derivatization of peptide 4. It is believed that this effect may be due to the hydrophobic interaction between aromatic chromophore and the protein. This phenomenon increases the efficient concentration of NQMP-biotin 1d conjugate at the protein surface thus shifting the position of photo-equilibrium in favor of 10a.

To demonstrate the reversibility of cysteine photo-derivatization, a 2 μM PBS solution of biotinylated BSA 10a was irradiated with 350 nm fluorescent lamp for 2 minutes and protein isolated and purified by spin filtration. Western blot analysis of the product showed complete removal of biotin tag from the protein (FIG. 6B, lane 4), while Coomassie Brilliant Blue staining confirmed presence of protein in the sample. The product protein has the same mobility as the 9a (FIG. 6B, lane 2) and biotinylated 10a, thus confirming clean regeneration of native BSA.

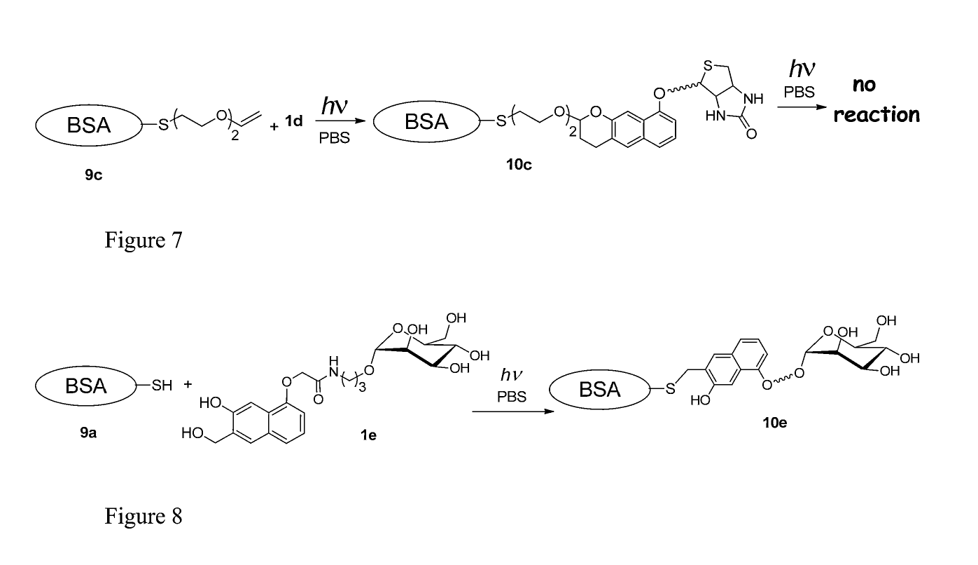

Permanent light-directed derivatization of cysteine residues can be accomplished via a two-step procedure. BSA (9a) is quantitatively converted into BSA-vinyl ether conjugate (9c) by the reaction with 2-(2-(vinyloxy)ethoxy)ethyl iodide. An 11 μM PBS solution of 9c in the presence of 100 μM of 1d was irradiated for 2 minutes with a 350 nm lamp in the formation of Diels-Alder adduct 10c (FIG. 7). Excess of reagent was removed by spin filtration and protein fraction washed several times. Western Blot analysis clearly indicates presence of BSA-biotin conjugate in the sample (FIG. 6A, lane 6). Quantification of protein and biotin in the resulting solution using both spectrophotometric and assay methods shows quantitative biotinylation of 9c (Table 3).

TABLE 3
Quantification of Bovine Serum Albumin Photo-labeling with 1d^{a)}

| | Protein | Total | | Yield of |
|---|---|---|---|---|
| | quantification | protein | | labeling |
| Substrate | Method | (μM) | Biotin (μM) | (%) |
| 10a | Bradford | 7.1 ± 0.2 | 7.0 ± 0.3^{b)} | 99 |
| 10a | Spectrophotometric | 7.5 ± 0.2 | 7.47 ± 0.07^{c)} | 99 |
| 10c | Bradford | 9.10 ± 0.05 | 9.57 ± 0.06^{b)} | 105 |
| 10c | Spectrophotometric | 9.3 ± 0.2 | 9.27 ± 0.03^{c)} | 99 |

^{a)}PBS solutions containing 0.1 mM of 1d and approximately 0.01 mM of protein were irradiated for 2 minutes at 350 nm at room temperature. Low molecular weight components were separated by ultrafiltration.
^{b)}Using an assay kit available under the trade designation FluoReporter Biotin Quantitation Assay Kit from Themo Scientific.
^{c)}Spectrophotometric determination of NQMP chromophore.

In contrast with 10a, irradiation of BSA-Biotin conjugate 10c, even for extended periods of time, does not result in the removal of a biotin tag (FIG. 6B, lane 7). In fact, electrophoretic mobility of BSA derivative 10c is not affected by irradiation illustrating photochemical stability of this conjugate.

Light directed NQMP-derivatization of BSA can be also employed for the preparation of glycoproteins. To illustrate this technique, a PBS solution of BSA (9a, 11 μM) and NQMP-mannose conjugate (1e, 100 μM) was exposed to 350 nm light for 2 minutes (FIG. 8). The resulting BSA-protein conjugate (10e) was isolated by spin filtration. The quantification of conjugated mannose indicated 95±1% yield of photo-glycosilation.

Reversible derivatization of the cysteine residues described in this disclosure can be useful for pulling down and purifying proteins containing or genetically engineered to contain solvent exposed cysteines. This approach has three important advantages over currently available methods: it releases native protein, with no remains of the linker used to immobilize the substrate; the release of the protein does not require any reagents, therefore producing pure substrate without any additional steps; the capture and release steps proceed in very high or quantitative yield. To illustrate the capabilities of the method, BSA has been quantitatively captured on neutravidin agarose gel, and then the protein has been photochemically cleaved from the beads.

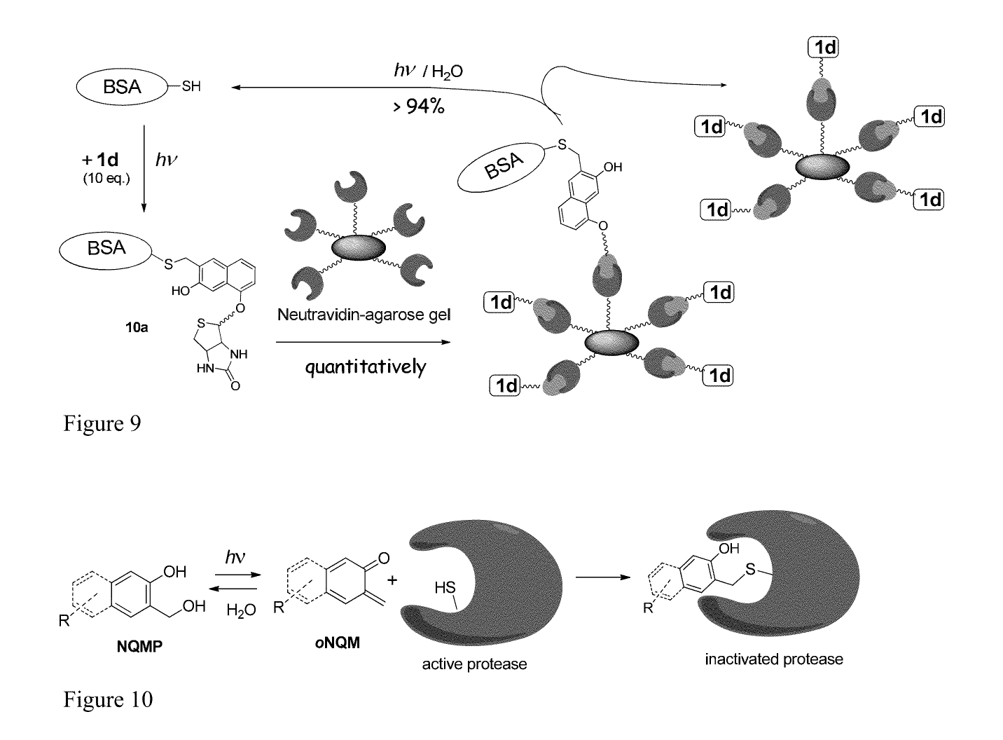

Following standard protocol (vide supra) a PBS solution containing 10 μM of BSA (9a) and 100 μM of NQMP-biotin conjugate 1d was irradiated for 5 minutes using 350 nm fluorescent lamp. NeutrAvidin Agarose resin (approximately 10% excess) was added to the photolysate and incubated in the dark for 30 minutes. The resulting resin was separated by centrifugation and washed. Combined supernatant contained no detectable amounts of BSA or 1d according to spectrophotometric analysis and Bradford assay. This observation indicates quantitative capture of BSA from the solution (FIG. 9).

The resulting NeutrAvidin Agarose resin was re-suspended in PBS buffer to achieve 10 μM concentration of bound BSA (and approximately 100 μM of bound 1d). The suspension was exposed to 350 nm light for 5 minutes under ambient conditions. The resin was removed by centrifugation and washed. Spectrophotometric and Bradford assay analyses showed 73-81% yield of BSA release (FIG. 9, Table 4). This is a noteworthy result as it illustrates significant reduction of BSA binding efficiency of immobilized NQMP. In fact, this data is similar to uncaging efficiency observed for peptide 5 and predicted by the equation 2. When BSA-derivatized NeutrAvidin Agarose resin suspension was diluted to approximately 5 μM concentration of bound protein (approximately 50 μM of 1d), 5 minutes of 350 nm irradiation resulted in 95% release of BSA (Table 4) Similar high yield of protein photo-cleavage from resin support was obtained at higher concentration when ethyl vinyl ether (equimolar amount in respect to 1d) was added to a suspension before irradiation (Table 4). This reagent apparently converts resin bound NQMP groups into photochemically stable benzochroman groups (vide supra).

TABLE 4
Photo-triggered release of BSA captured on NeutrAvidin Agarose gel.^{a}

| BSA | | | |
|---|---|---|---|
| concentration | Protein | Concentration of | |
| in the gel | quantification | ethyl vinyl ether | Yield of BSA |
| (μM) | method | (μM) | release (%) |
| 10 | Bradford | 0 | 81 ± 1 |
| 10 | Spectrophotometric | 0 | 73 ± 2 |
| 5 | Bradford | 0 | 95 ± 2 |
| 5 | Spectrophotometric | 0 | 95 ± 2 |
| 10 | Bradford | 100 | 95 ± 3 |
| 10 | Spectrophotometric | 100 | 94 ± 2 |

^{a}NeutrAvidin Agarose resin suspensions were irradiated for 5 minutes at 350 nm at room temperature.

### Conclusions

A photochemical method for the selective derivatization of free cysteine residues in peptides and proteins has been demonstrated. This technique is based on a very facile addition of thiols to o-naphthoquinone methides (oNQMs) resulting in the formation thioethers. In aqueous solution endogenous nucleophiles other thiols are not reactive enough to compete with hydration of oNQM. oNQMs are generated by efficient dehydration of 3-(hydroxymethyl)-2-naphthols (NQMPs) upon exposure to 300 or 350 nm light. The utility of this method has been demonstrated by derivatizing bovine serum albumin (BSA) with biotin and mannose. Any other substrate, which does not decompose during short exposure to 350 nm light, can be conjugated with cysteine residues via this technique. Exhaustive derivatization of all available cysteines requires the concentration of NQMP-TAG reagent to exceed substrate concentration by at least 300 μM. It is important to note that NQMP-TAG solutions employed for the derivatization can be re-used numerous times because all unreacted oNQM molecules undergo hydration to regenerate starting NQMP-TAG. Very short life time of oNQM in aqueous solutions (approximately 7 ms) makes this labeling technique suitable for time-resolved studies, for example for taking “instant pictures” of the exposed cysteines in folding protein by using laser flash irradiation of the sample with various delays.

The NQMP thioether can be later cleaved by repeating irradiation in a dilute solution (40 μM or lower) in the absence of free NQMP-TAG. Quantitative release of NQMP-caged substrates at any concentration can be achieved by introducing ethyl vinyl ether before photolysis, which converts NQMP into a photo-stable benzochroman. Reversible biotinylation of BSA, as well as capturing on NeutrAvidin Agarose resin and then cleaving the protein from the beads has been demonstrated. This procedure allows for quantitative capture and for traceless release of protein in a high yield. The cleavage of protein from the solid support does not require any reagents, simplifying purification. Reversible modification of the cysteine residues in proteins can be employed for the photoregulation of protein activity either by caging of catalytically active cysteine (e.g., in cysteine proteases) or by attaching activity-modifying fragment to it. Reversible PEGylation of active peptides and proteins can be employed for light-directed drug delivery or time-resolved activation of these substances.

### Example 2

### Reversible Photochemical Inhibition of Cysteine Proteases

The technology for the photochemical inhibition of cysteine proteases has been developed. Irradiation of aqueous solution containing cysteine protease and 3-(hydroxymethyl)-2-naphthol derivatives (NQMP) results in conversion of free cysteine residues in protein into NQMP-thioether. This can render the protease inactive. Subsequent re-irradiation of NQMP-caged proteases in the absence of NQMP reagent can restore the activity of the protease. Alternatively, restoration of cysteine protease activity can be achieved in the presence of NQMP if vinyl ether is employed as NQMP-trapping reagent.

It has been shown that 2-napthoquinone-3-methides (oNQMs) photochemically generated from 3-(hydroxymethyl)-2-naphthol derivatives (NQMP) selectively react with solvent-exposed cysteine residues in peptides and proteins. Here, this chemistry for the reversible caging of cysteine residue in cysteine proteases (FIG. 10) has been employed. An aqueous solution of active protease and excess of NQMP is irradiated with 350 nm fluorescent lamp. Depending on the duration of irradiation and the concentration of NQMP the activity of the enzyme can be reduced or completely suppressed. The protease inhibition is apparently due to the formation of a thioether in reaction between photochemically generated oNQM and catalytically-active cysteine residue in an active site of the enzyme.

Using experimental rates of oNQM hydration and reactions with simple thiols (Arumugam et al., J. Am. Chem. Soc. 2009, 131:11892), as well as photochemical efficiencies of NQMP and NQMP-SR reactions (Arumugam et al., J. Am. Chem. Soc. 2009, 131:11892; and Arumugam et al., J. Am. Chem. Soc. 2011, 133:5573), it can be determined that 90+% inhibition of a protease activity is achieved at starting NQMP concentration equal [NQMP]=300 μM+[Protease]. In real experiments even lower concentration of NQMP produced complete inhibition of papain activity.

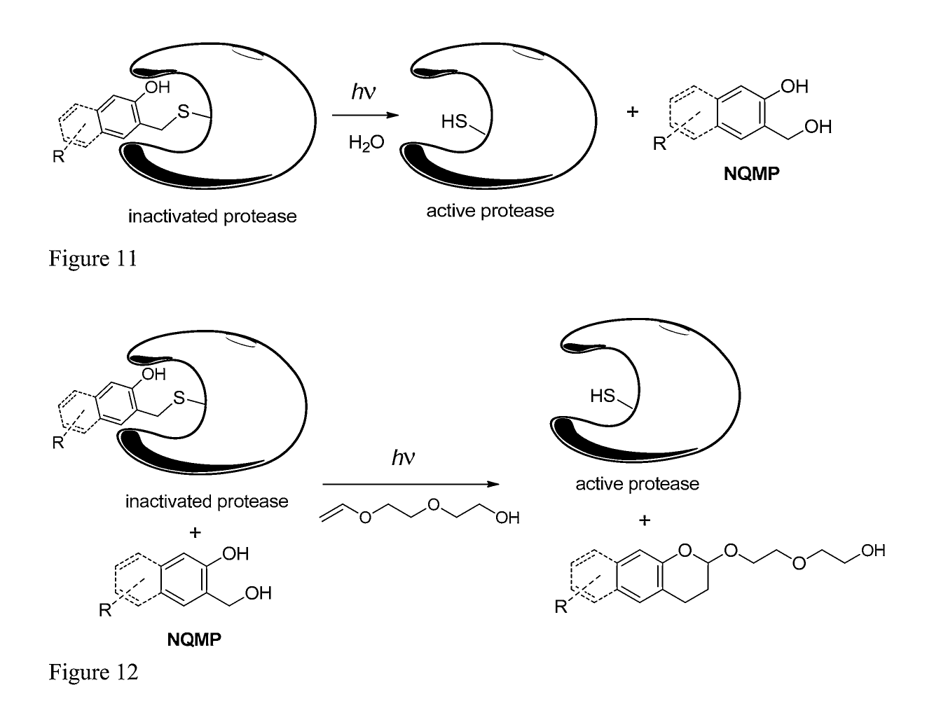

The photochemically inhibited protease remains inactive even after prolonged incubation in PBS solution in the presence of 5 mM of cysteine. This observation indicates that thioether blocking the cysteine residue of the catalytic diad is hydrolytically stable. The NQMP thioethers can be quantitatively cleaved upon 300 or 350 nm photolysis in 10% quantum yield (Arumugam et al., J. Am. Chem. Soc. 2011, 133:5573). Thus, subsequent irradiation of NQMP-inhibited protease in an aqueous solution containing no NQMP reagents results in the re-activation of the substrate. This process apparently regenerates free cysteine residue in active site of the protease (FIG. 11).

In essence, the formation of NQMP-thioether is a photochemically driven equilibrium between a cysteine residue and NQMP on one side and NQMP-thioether and water on the other. Since oNQM reacts about five orders of magnitude faster with thiols (k_{RSH} is approximately 2.2×10⁵ M⁻¹ seconds⁻¹) (Arumugam et al., J. Am. Chem. Soc. 2009, 131:11892) than with water (k_{H2O} is approximately 2.6 M⁻¹ seconds⁻¹) (Arumugam et al., J. Am. Chem. Soc. 2009, 131:11892) and NQMP-thioethers are 50% less prone to photoelimination than NQMP itself (Arumugam et al., J. Am. Chem. Soc. 2011, 133:5573), equilibrium is shifted towards the formation of inhibited form of the enzyme. However, if no NQMP is present in the solution, irradiation of inactive protease results in the cleavage of the NQMP-thioether and the restoration of enzyme activity.

Photochemical restoration of the NQMP-caged cysteine protease activity can be achieved even in the presence of NQMP reagent when vinyl ether derivatives are employed as oNQM trapping agents. Very facile Diels-Alder addition of vinyl ether to intermediate oNQM converts the latter into a photostable benzochroman (Arumugam et al., J. Am. Chem. Soc. 2009, 131:11892) (FIG. 12). This reaction efficiently removes oNQM precursors from the reaction mixture and shifts photo-equilibrium towards substrates with free cysteine residue.

Reversible photochemical inhibition of cysteine proteases should not be limited to NQMP. Any analog of o-hydroxybenzyl alcohol that produces quinone methide upon irradiation will form thioether with free cysteine. NQMP has two main advantages over other o-hydroxybenzyl alcohol analogs: it can be activated with 350 nm light and it has high selectivity towards thiols. Quinone methide-based inhibition could be extended to serine and threonine proteases.

### Experimental

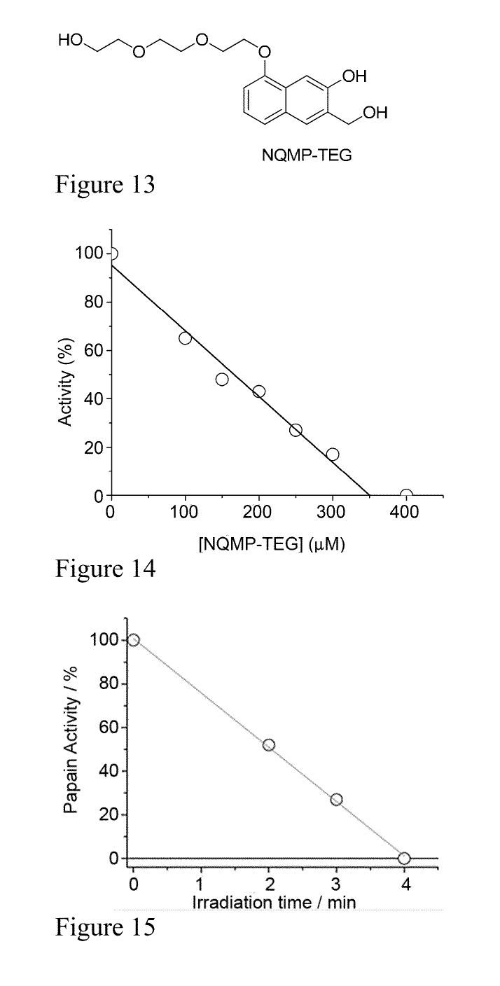

Papain from papaya latex was employed to demonstrate this technology. Papain was activated by incubation the enzyme with 100-40 fold excess of free cysteine for 45 minutes in PBS solution. The concentration of the papain in the stock solution was determined by its abortion at 280 nm (s=57600 M⁻¹ cm⁻¹) (Pace et. al., Protein Sci. 1995, 4:2411-2423). The activity of the protease was measured using N-α-benzoyl-L-arginine-p-nitroanilide (L-BAPNA). The enzyme cleaves the amide bond of L-BAPNA releasing p-nitroaniline (pKa=1.0) (Wang et al., J. Chromat. A. 2002, 979:439-446). The formation of the latter can be followed by the rise of characteristic absorbance at 410 nm (ε=9800). Water-soluble NQMP-TEG (FIG. 13) was employed for inhibition experiments.

### Photochemical Inhibition of Papain Using Different Concentrations of NQMP.

A PBS solution containing 1.7 μM of papain, 80 μM of cysteine and variable amounts of NQMP-TEG was irradiated for 2 minutes with 350 nm fluorescent lamps. A stock solution of BAPNA was then added to a photolysate to achieve 800 μM concentration of the substrate and rate of hydrolysis was measured (Table 5, FIG. 14).

TABLE 5
Inhibition of Papain Activity by Various Concentration of NQMP-TEG

| | | Irradiation | |
|---|---|---|---|
| | [NQMP-TEG]/ | time/ | Relative |
| | μM | minutes | activity |
| 1 | 0 | 0 | 100% |
| 2 | 0 | 2 | 100% |
| 3 | 100 | 2 | 65% |
| 4 | 150 | 2 | 48% |
| 5 | 200 | 2 | 43% |
| 6 | 250 | 2 | 27% |
| 7 | 300 | 2 | 17% |
| 8 | 400 | 2 | 0% |

The data indicates that about 350 μM of NQMP-TEG is required to completely inhibit the activity of Papain at 2 minutes of irradiation. MALDI-TOF mass spectral analysis of native papain and inhibited papain showed mass increase corresponding to an addition of a single NQMP-TEG unit to papain.

### Photochemical Inhibition of Papain Using Different Irradiation Time.

A PBS solution containing 0.91 μM of papain, 44 μM of cysteine and 109 μM of NQMP-TEG was irradiated for 2, 3 or 4 minutes with 350 nm fluorescent lamps. A stock solution of BAPNA was then added to a photolysate to achieve 200 μM concentration of the substrate and rate of hydrolysis was measured (Table 6, FIG. 15).

TABLE 6
Activity of Papain Vs different irradiation time.

| | Irradiation | |
|---|---|---|
| | time/ | Relative |
| | minutes | activity |
| 1 | 0 | 100% |
| 2 | 2 | 52% |
| 3 | 3 | 27% |
| 4 | 4 | 0% |

### Restoration of Papain Activity.

A sample of deactivated papain was prepared as described above. The protein was separated from low molecular weight compounds using gel filtration (Sephadex PD-10 column). To check for the completeness of inhibition an excess of cysteine was added to a PBS solution of the inhibited papain and incubated for 45 minutes. The resulting solution does not cleave L-BAPNA indicating that protease activity has not been restored by the addition of cysteine. This solution was then re-irradiated with 350 nm fluorescent lamps for 15 minutes. This treatment resulted in the restoration of papain activity to about 85-92% of the wild type papain.

Alternatively, re-activation of papain was achieved without isolation of inhibited enzyme. Ethyl vinyl ether derivative, 2-(2-(vinyloxy)ethoxy)ethanol, was added to a photolysate containing inhibited papain, NQMP-TEG, and cysteine in PBS solution. The amount of vinyl ether added was equal to the initial amount of NQMP-TEG. Irradiation of the resulting solution with 350 nm light also resulted in the restoration of activity (Table 7).

TABLE 7
Photochemical Re-Activation of Papain

| Irradiation | |
|---|---|
| time/ | Papain |
| minutes | Activity |
| 0 | 0% |
| 3 | 28% |
| 4 | 40% |
| 5 | 47% |
| 7 | 58% |
| 8 | 76% |

### Example 3

### General Information

All organic solvents were dried and freshly distilled before use. Flash chromatography was performed using 40-63 μm silica gel. All NMR spectra were recorded on 400 MHz instruments in CDCl₃ and referenced to TMS unless otherwise noted. Solutions were prepared using HPLC grade water, and acetonitrile. Photolyses were carried out using mini-Rayonet photochemical reactor equipped with 8 fluorescent UV lamps (4W, 300 or 350).

### Materials:

1-iodo-2-(2-(2-methoxyethoxy)ethoxy)ethane (Arumugam et al., J. Am. Chem. Soc. 2011, 133:15730), 2-(2-(Vinyloxy)ethoxy)ethyl tosylate, 6-(hydroxymethypnaphthalene-1,7-diol (NQMP 1b; Arumugam et al., J. Am. Chem. Soc. 2011, 133:5573), 8-TEG-3-(hydroxymethyl)naphthalen-2-ol (NQMP-TEG 2c), 8-(Biotin-TEG)-3-(hydroxymethyl)naphthalen-2-ol (NQMP 1d), and 1-α-(3-Azidopropyl)-2,3,4,6-tetra-O-acetyl-D-mannopyranoside (S2; Hayes et al., Tetrahedron 2003, 59:7983), were prepared following the previously reported procedures. Fmoc-L-amino acid derivatives and resins were purchased from NovaBioChem and Applied Biosystems; peptide synthesis grade DMF was obtained from EM Science and N-methylpyrrolidone (NMP) from Applied Biosystems; Bovine Serum Albumin (BSA) was acquired from Thermo Scientific.

2-(2-(vinyloxy)ethoxy)ethyl iodide (S1):

NaI (1.5 g, 10 mmol) was added to a solution of 2-(2-(vinyloxy)ethoxy)ethyl tosylate (1.43 g, 5 mmol) in acetone (20 mL) and reaction mixture was refluxed for 8 h. Solvent was removed under reduced pressure, the residue was dissolved in dichloromethane, washed with brine, dried over anhydrous magnesium sulfate, and the solvent was removed in vacuum. The product was purified by chromatography (60% EtOAc in hexanes) to yield 0.99 g (82%) of S1 as a yellowish oil. ¹H NMR: 6.40 (dd, J=14.3, 6.8 Hz, 1H), 4.10 (dd, J=14.3, 2.0 Hz, 1H), 3.94 (dd, J=6.8, 2.0 Hz, 1H), 3.81-3.73 (m, 2H), 3.73-3.63 (m, 4H), 3.18 (t, J=7.0 Hz, 2H). ¹³C NMR: 151.95, 87.18, 72.37, 69.49, 67.54, 2.91. FW calc. (C6H11IO2): 241.9804, EI-HRMS: 241.9810.

### Octapeptides Ac-Cys-Lys-Tyr-Trp-Gly-Arg-Asp-Ser-NH₂ (4) and Ac-Met-Lys-Tyr-Trp-Gly-Arg-Asp-Ser-NH₂ (6)

(4) and (6) were synthesized following a standard Fmoc chemistry protocol on Rink amide AM resin (0.25 mmol). Peptides preparation was accomplished on a Applied Biosystems ABI 433A peptide synthesizer equipped with UV-detector using N^{α}-Fmoc-protected amino acids and 2-(1H-bezotriazole-1-yl)-oxy-1,1,3,3-tetramethyl hexafluorophosphate (HBTU)/1-Hydroxybenzotriazole (HOBt) as the activating reagents. The resin was treated with cleavage solution of 95:2.5:2.5 TFA/water/TIS (5 mL) for 1 hour. The resin was filtered, washed with TFA (5 mL), and the filtrate was then concentrated in vacuum to approximately ⅓ of its original volume. The peptide was precipitated by using diethyl ether (0° C., 100 mL) and recovered by centrifugation at 3000 rpm for 15 minutes. The crude peptide was purified by RP-HPLC on a semi-preparative C-18 column using a linear gradient solvent (0-95% CH₃CN in H₂O, 0.1% TFA in 40 minutes; flow: 1 mL/minute) and the appropriate fractions were lyophilized to afford target peptide. Peptide 4: FW Calcd. for C₄₈H₇₀N₁₅O₁₄S⁺(M+H⁺) 1112.4942, MALDI HRMS found 1112.6621. Peptide 6: FW Calcd for C₄₉H₇₂N₁₅O₁₄S⁺ (M+H⁺) 1126.5098. MALDI HRMS found 1126.5098.

Peptide 4 dimer [Ac-Cystine-Lys-Tyr-Trp-Gly-Arg-Asp-Ser-NH₂]₂ (7):

Peptide 4 (40 mg) was dissolved in 2:8 DMSO/H₂O (4 ml) and stirred at room temperature for 2 days. The dimer was purified by RP-HPLC on a semi-preparative C-18 column using a linear solvent gradient (0-95% CH₃CN in H₂O, 0.1% TFA in 40 minutes; flow: 1 mL/minute). Appropriate fractions were lyophilized to afford the target peptide 7. F.W. Calcd. for C₉₆H₁₃₇N₃₀O₂₈S₂⁺ (M+H⁺): 2221.9655; MALDI HRMS found: m/z 2221.8560.

Preparation of NQMP-D-mannopyranoside conjugate 1e.

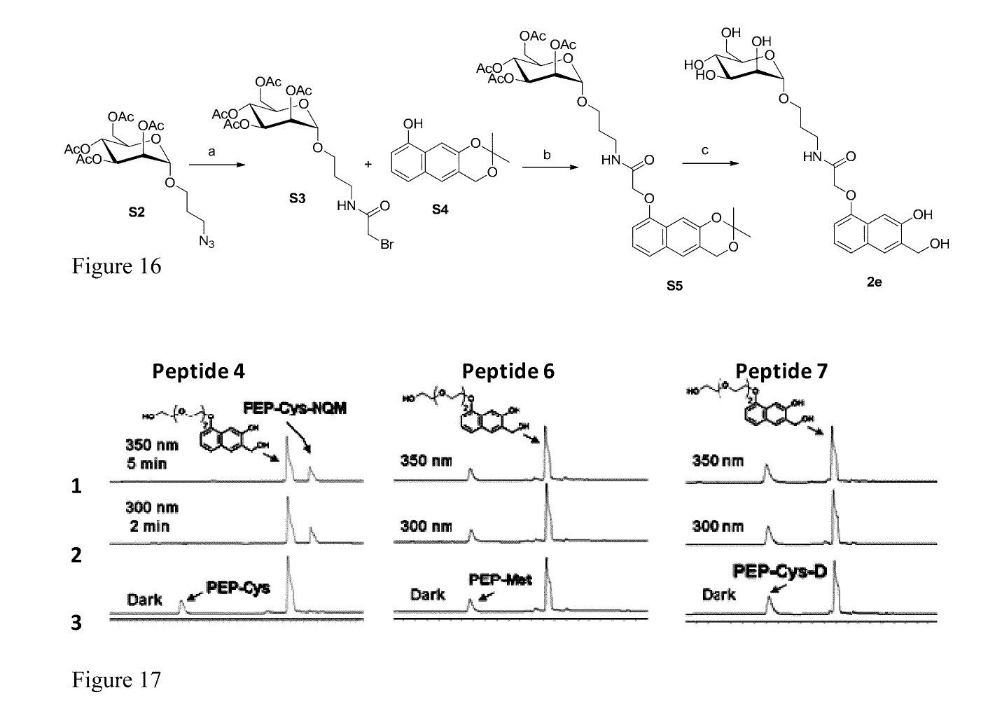

The preparation of NQMP-D-mannopyranoside conjugate 1e is schematically illustrated in FIG. 16. Reagents and conditions: (a) i) H₂, Pd/C, MeOH; ii) BrCH₂COCl, Pyridine, DCM; (b) S4, NaOH, DMF; (c) i) 7N NH₃ in MeOH; ii) 2:8 H₂O/AcOH

1-α-(3-Bromoacetyl-propyl)-2,3,4,6-tetra-O-acetyl-D-mannopyranoside (S3).

Palladium hydroxide (20 wt % Pd on carbon, 0.11 g) was added to a solution of 1-α-(3-azidopropyl)-2,3,4,6-tetra-O-acetyl-D-mannopyranoside (S2, 0.431 g. 1.0 mmol) in MeOH (40 ml). The mixture was stirred under H₂ for 2 hours. Solids were removed by filtration; the solvent was removed under reduced pressure. The crude material was dissolved in dry CH₂Cl₂ and bromoacetyl chloride (0.21 ml, 2.0 mmol), Et₃N (0.558 ml, 4.0 mmol) were added. The reaction mixture was stirred at room temperature for 2 hours and solvent was removed under reduced pressure. The crude product was purified by flash chromatography (1:2 to 1:0 EtOAc/CH₂Cl₂) to give S3 (0.280 g, 53%) as brownish oil. ¹H NMR (300 MHz): 6.81 (br, 1H, NH), 5.26-5.12 (m, 3H), 4.73 (s, 1H, H-1), 4.19 (dd, 1H, J=5.3, 12.3 Hz, H-5), 4.00 (m, 1H, H-5′), 3.89 (m, 1H), 3.79 (s, 2H, BrCH₂), 3.72 (m, 1H, NHCH₂), 3.44 (m, 1H, NHCH₂), 3.32 (m, 2H, OCH₂CH₂), 2.06 (s, 3H, Ac), 2.00 (s, 3H, Ac), 1.95 (s, 3H, Ac), 1.90 (s, 3H, Ac), 1.79 (m, 2H, OCH₂CH₂). ¹³C NMR (75 MHz): 170.74, 170.12, 169.79, 166.05, 97.80, 69.56, 69.15, 68.67, 66.65, 66.28, 62.66, 38.08, 29.22, 28.92, 20.96, 20.86, 20.80, 20.77. FW Calcd for C₁₉H₂₈BrNNaO₁₁⁺ (M+Na⁺): 548.0738; MALDI HRMS found: m/z 548.0668.

Protected 1-α-(3-(NQMP-acetyl)propyl)-2,3,4,6-tetra-O-acetyl-D-mannopyranoside (S5).

NaOH (12.0 mg, 0.3 mmol) was added to a solution of S4 (69.1 mg, 0.3 mmol) in methanol (10 ml). After 10 minutes at room temperature solvent was removed under reduced pressure and the resulting salt re-dissolved in DMF (20 ml). A solution of bromide S3 (131.6 mg, 0.25 mmol) in DMF (10 ml) was added to the reaction mixture and it was stirred at room temperature for 4 hours. The solvent was removed under reduced pressure. And the residue purified by flash chromatography (1:0 to 4:1 CH₂Cl₂/EtOAct) to give S5 (160 mg, 95%) as yellowish oil. ¹H NMR (300 MHz): 7.51 (s, 1H), 7.41 (s, 1H), 7.33 (d, 1H, J=8.5 Hz), 7.16 (t, 1H, J=7.9 Hz), 6.79 (t, 1H, J=6.1 Hz, NH), 6.69 (d, 1H, J=7.3 Hz), 5.28-5.17 (m, 3H), 5.01 (s, 2H), 4.75 (s, 1H, H-1), 4.61 (s, 2H), 4.24 (dd, 1H, J=5.3, 12.3 Hz, H-5), 4.05 (m, 1H, H-5′), 3.95 (m, 1H), 3.72 (m, 1H), 3.44 (m, 3H), 2.10 (s, 3H, Ac), 2.03 (s, 3H, Ac), 1.98 (s, 3H, Ac), 1.92 (s, 3H, Ac), 1.86 (t, 2H, J=6.3 Hz, OCH₂CH₂), 1.56 (s, 6H). ¹³C NMR (75 MHz): 170.79, 170.15, 169.98, 169.91, 168.66, 152.18, 150.03, 129.71, 125.83, 123.73, 122.08, 121.48, 106.44, 105.83, 100.19, 97.96, 69.74, 69.25, 68.70, 68.04, 66.44, 66.25, 62.78, 61.26, 36.55, 29.74, 25.17, 25.12, 21.05, 20.91, 20.87, 20.83. FW Calcd for C₃₃H₄₁NNaO₁₄⁺ (M+Na⁺): 698.2419; MALDI HRMS: m/z 698.2216.

### 1-α-(3-(NQM-acetyl)-propyl)-D-mannopyranoside (S6).

S5 was dissolved in 7N metanolic ammonia (20 ml) at 0° C. and stirred at room temperature for 12 hours. The solvent was removed under reduced pressure, residue re-dissolved in 2:8 H₂O/HOAc (20 ml), and stirred at room temperature for 2 hours. The solvent was removed under reduced pressure and the residue was purified by flash chromatography on Iatrobeads (10-20% MeOH in CH₂Cl₂) to give S6 (61 mg, 65%) as colorless syrup. ¹H NMR (300 MHz, CD₃OD): 7.75 (s, 1H), 7.54 (s, 1H), 7.38 (d, 1H, J=8.2 Hz), 7.15 (t, 1H, J=7.9 Hz), 6.76 (d, 1H, J=7.3 Hz), 4.80 (s, 2H), 4.70 (d, 1H, J=1.5 Hz, H-1), 4.65 (s, 2H), 3.86-3.48 (m, 6H), 3.46-3.28 (m, 4H), 1.82 (m, 2H). ¹³C NMR (75 MHz, CD₃OD): 170.18, 153.45, 152.39, 131.03, 129.77, 126.29, 126.19, 122.50, 121.33, 105.76, 103.13, 100.52, 73.54, 71.47, 70.95, 67.79, 67.50, 64.96, 61.78, 60.06, 36.40, 29.16. FW Calcd for C₂₂H₂₉NNaO₁₀⁺ (M+Na⁺) 490.1684; MALDI HRMS: m/z 490.1181

### BSA-Vinyl Ether Conjugate (9c).

S1 (1.92 mg, 8 mmol) was added to a solution of BSA (53 mg, ca 0.8 μmol) in 3 mL of 0.1N phosphate buffer (pH=8)+0.5 mL of acetonitrile and gently shaked using mechanical shaker for 12 hours at room temperature. Ellman's test shows complete conversion of free thiol group in BSA. Aqueous layer was washed with ethyl acetate and was freeze-dried to produce 9b (63 mg—includes some phosphate salt). The modified BSA 9b was further purified by multiple spin filtration.

### BSA-TEG-Me Conjugate (9b).

BSA-TEG-Me conjugate (9b). was obtained using a similar procedure but 1-iodo-2-(2-(2-methoxyethoxy)ethoxy)ethane instead of S1.

### Procedures

### Photochemical Labeling of Peptides 4, 6, and 7.

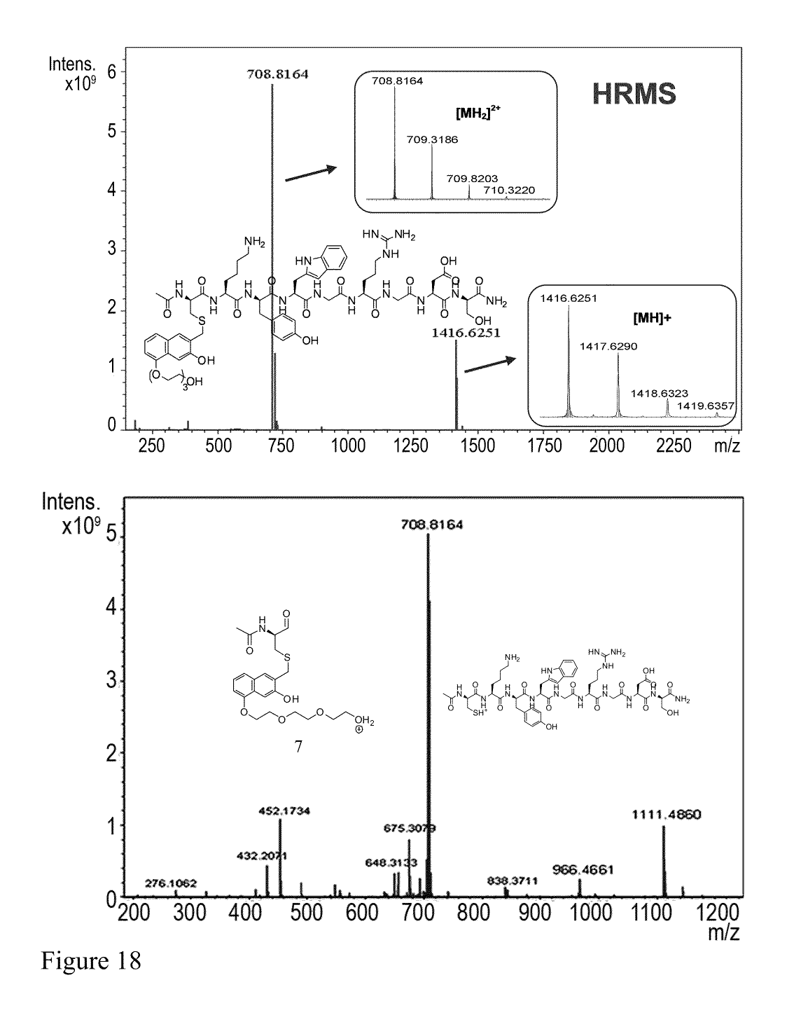

0.1 mM PBS solutions of peptides 4, 6, and 7 were irradiated with 300 or 350 nm fluorescent lamps in the presence of variable amounts of NQMP-TEG (2c, 0.1-0.4 mM). Composition of the photolysates was analyzed using HPLC (FIG. 17). Peptide 4-NQMP-TEG conjugate (5) was isolated using semi-prep C-18 HPLC column and characterized by ESI-HRMS (FIG. 18). The concentration of labeled peptide 5 was determined by UV spectroscopy using characteristic 330 nm band of NQMP chromophore. This solution was then used in calibration of HPLC instruments.

### Control Experiments:

0.1 mM PBS solutions of NQMP-TEG (2c) and peptide 4 were irradiated separately for 12 minutes using both 300 and 350 nm fluorescent lamps. No decomposition or losses of materials were detected by HPLC.

### General Procedures for Photochemical Biotinylation of Proteins.

Solutions of BSA 9a, its derivatives 9b,c (3.6 mg in 5 mL of PBS buffer; 11 μm), and NQMP-biotin (1d, 100 μM) were irradiated with 350 nm fluorescent lamp for 2 minutes. The excess of reagent was removed by spin filtration.

Derivatization of BSA with Mannose.

Solutions of BSA (9a, 11 μM in PBS buffer), and NQMP-mannose (1e, 100 μM) were irradiated with 350 nm fluorescent lamp for 2 minutes. The excess of reagent was removed by spin filtration.

Photochemical Removal of the Biotin Tag from BSA Derivative 10a.

A solution of BSA-biotin conjugate 10a (2 μM PBS buffer) was irradiated with 350 nm fluorescent lamp for 2 minutes. Protein was isolated by spin filtration.

### Spectrophotometric Measurements of BSA (9b and 9a) Concentration.

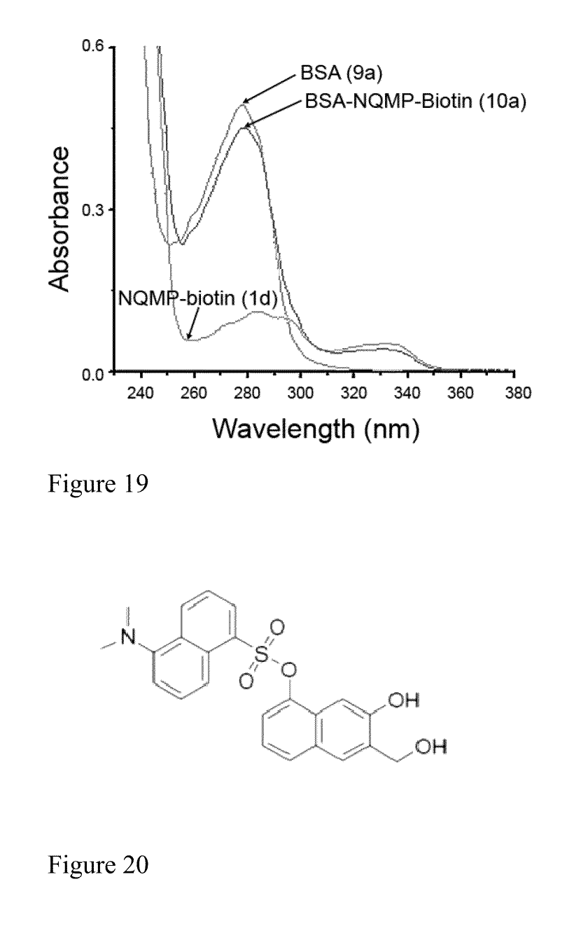

9b quantification was conducted by measuring absorbance of 9b solution at 278 nm. This wavelength corresponds to the maxima of a strong BSA band (ε=49,200; FIG. 19).

258 nm and 331 nm wavelengths were employed for separate quantification of NQMP and BSA chromophores in protein 10a solutions. At 258 nm NQMP chromophore has low extinction coefficient (ε=5,600 FIG. 19), while BSA shows much stronger absorbance (ε=28,900). NQMP has a weak band with maximum at 331 nm (ε=5,200), where BSA has virtually no absorbance (ε=180, FIG. 19).

### Quantification of the Protein Amount Using Bradford Protein Assay.

Different concentrations of protein solutions were mixed with Coomassie Blue solution (Bio-Rad) The absorption of the resulting solution at 584 nm was measured in a multiwell microplate using BMG Labtech POLARstar Optima reader. The amount of protein is analyzed by comparing the absorption to a protein standard curve.

### Biotin Quantification in the Sample of BSA-Biotin Conjugates 10a and 10b.

The amount of conjugated biotin was quantified by using Fluorescence Biotin Quantitation Kit (Thermo Scientific). Briefly, the fluorescence intensity of the dye of avidin significantly increases when the weakly interacting quencher HABA (4′-hydroxyazobenzene-2-carboxylic acid) is displaced by the biotin. The premix fluorescence dye labeled avidin with HABA (DyLight Reporter) is added to the protein solution containing conjugated biotin. Because of its higher affinity for avidin, biotin displaces the HABA, allowing the avidin to fluoresce. The fluorescence intensity was measured on a BMG Labtech POLARstar Optima reader. The amount of biotin is measured in a microplate by comparing the fluorescence to a biotin standard curve.

### Quantification of Conjugated Mannose

Quantification of conjugated mannose in the sample of BSA-mannose conjugate 11 was performed on DIONEX ICS-3000 HPAEC chromatograph using deionized water and 200 mM NaOH as an eluent. Sample preparation: 1-2 mg of mannose-conjugated BSA sample and standard D-(+)-mannose were treated with 2 M TFA in water (250 μL) for 4 hours at 100° C. Sample and standard were spin dried, re-dissolved in water (500 μL) and filtered. Sample concentration was then determined based on the calibration curves of mannose standards.

### Western Blot Analysis.

The photo-derivatized BSA samples (25 μg of protein per lane) were resolved on a 4-20% SDS-PAGE gel (Bio-Rad) and transferred to a nitrocellulose membrane. Next the membrane was blocked in blocking buffer (nonfat dry milk (5%; Bio-Rad) in PBST (PBS containing 0.1% Tween-20 and 0.1% Triton X-100)) for 2 hours at room temperature. The blocked membrane was then incubated for 1 hour at room temperature with an antibiotin antibody conjugated to horseradish peroxidase (HRP) (1:100000; Jackson ImmunoResearch Lab, Inc.) in blocking buffer and washed with PBST (4×10 minutes). Final detection of HRP activity was performed using ECL Plus chemiluminescent substrate (Amersham), exposure to film (Kodak) and development using a digital X-ray imaging machine (Kodak). Coomassie Brilliant blue staining was used to confirm total protein loading.

### Photo-Triggered Catch and Release of BSA Using NeutrAvidin Agarose Gel.

200 μL of PBS solution containing 0.01 mM of BSA and 0.1 mM of NQMP-biotin conjugate (1d) were irradiated for 5 minutes using 350 nm fluorescent lamps. 70 μL, of high capacity Neutravidin Agarose resin (Thermo Scientific) was mixed in using vortex and the mixture was incubated in dark for 30 minutes. The suspension was then centrifuged and the supernatant was removed. No BSA can be detected in the supernatant solution by spectroscopic or Bradford assay method. The derivatized Agarose gel was washed fresh PBS solution (3×200 μL). The washes contain no detectable amounts of NQMP-biotin conjugate 1d. Neutravidin Agarose gel was re-suspended in 200 μL of PBS buffer and irradiated for 5 minutes using 350 nm fluorescent lamps. The gel was separated from the aqueous phase by centrifugation and washed with PBS (3×250 μL). The combined supernatant was analyzed for released BSA by spectrophotometric and Bradford assay methods.

The procedure was repeated with two modifications: (A) the second irradiation was conducted in 400 μL of PBS; and (B) the second irradiation was conducted in 200 μL of PBS but in the presence of 0.1 mM of ethyl vinyl ether.

### Example 4

### BSA Labeling with DANSYL Group

500 μL of 68 μM solution of BSA in PBS and 250 μL of 1 mM solution NQMP-Dansyl conjugate (NQMP-DNS, FIG. 20) in PBS with 16% of DMSO were added to 1.75 mL of PBS (pH=7.1). The solution was irradiated in mini-Rayonet photoreactor equipped with 350 nm fluorescent tubes for 6 minutes. The low molecular weight components were removed from photolysate using sephadex-G25 PD-10 column. Three fractions of the eluent were collected and analyzed separately. Dansyl and BSA contents were determined using spectroscopic method (Table 8; 1 mL cell; calibration plots at 278 nm and 337 nm were constructed using solutions of BSA and NQMP-DNS). Average Dansyl to BSA ratio was 1.77±0.30.

TABLE 8
Spectroscopic determination of BSA and
Dansyl contents of NQMP-DNS labeled BSA.

| | Absorbance | Absorbance | | | NQMP/ |
|---|---|---|---|---|---|
| | at 278 nm | at 337 nm | [Dansyl]/M | [BSA]/M | BSA |
| 1 | 0.368 | 0.042 | 1.1E−05 | 7.8E−06 | 1.4 |
| 2 | 0.626 | 0.089 | 2.3E−05 | 1.3E−05 | 1.9 |
| 3 | 0.261 | 0.052 | 1.0E−05 | 5.2E−06 | 2.0 |

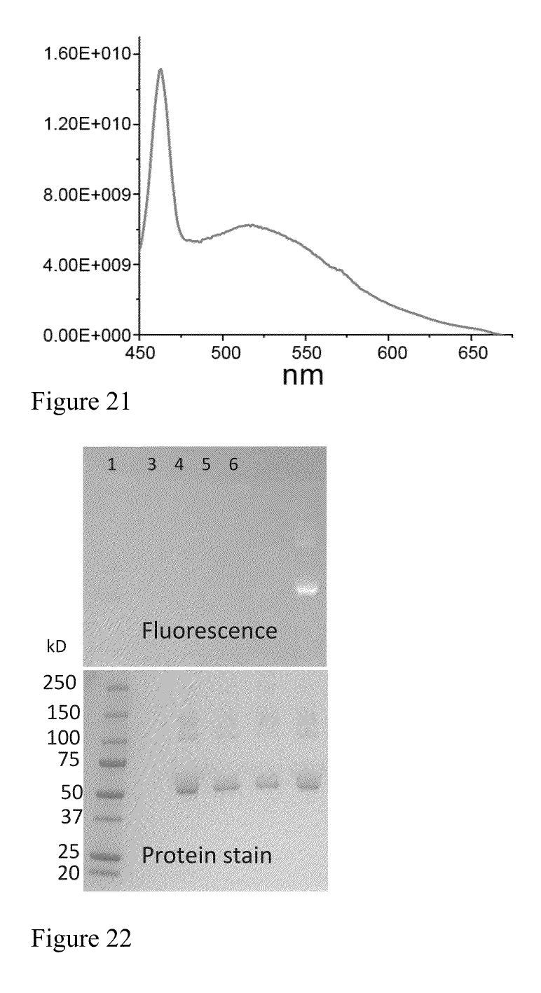

The fluorescent spectrum of Dansyl-BSA conjugate recorded with 400 nm excitation contains two intense bands at 463 nm and 518 nm (FIG. 21). The first band observed in a BSA-only solution, while the second belongs to Dansyl chromophore.

The control dark experiment, in a stark contrast to the NQMP-Biotin, shows significant non-specific binding of NQMP-DNS to BSA. PBS solutions containing 11 μM of BSA and 100 μM of NQMP-DNS were incubated in the dark for 30 and 90 minutes at room temperature. The low molecular weight components were removed from the reaction mixture using PD-10 column, and Dansyl to BSA ratios were determined spectroscopically. The average ratio was 0.41±0.07 after 30 minutes and 0.43±0.15 after 90 minutes of incubation. This non-specific binding is not surprising since dansyl-amide is known to have significant affinity to BSA binding site I (Jisha et al., J. Am. Chem. Soc. 2006, 128:6024). It is interesting to note that subtracting fraction of non-specifically bound NQMP-DNS from total dansyl content brings dansyl to BSA ratio close to biotin per BSA ratio. It also important to note that non-specific dansyl labeling does not survive SDS-PAGE (vide infra).

PBS solutions containing 12 μM of BSA and 100 μM of NQMP-DNS were irradiated with 350 nm lamps or incubated in the dark for 30 and 90 minutes (vide supra). The unconjugated NQMP-DNS was removed by passing the samples through a PD-10 column and fractions collected were further filtered and concentrated using a centrifugal device available under the trade designation 10K MWCO NANOSEP from Life Sciences. The samples were reconstituted in PBS (pH 7.4) and the total protein content was assessed using the bicinchonic acid assay (BCA; Pierce Biotechnology) and stored at 4° C. until required for use. The BSA samples (1.2 uM) were added to native sample buffer and resolved using a 4-20% Tris-HCl gel (Bio-Rad) without boiling (FIG. 22). The gel was imaged using a ChemiDoc MP imaging system (Bio-Rad). The fluorescence readout has been performed using 302 nm excitation and 548-630 nm filter (Amber or Ethidium bromide filter) in the recording channel. Coomassie staining of the gel was done to confirm total protein loading. Only irradiated samples show fluorescence at greater than 500 nm.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claim

What is claimed is:

1. A method for labeling a protein having a thiol group, the method comprising:
generating an o-naphthoquinone methide; and
contacting the o-naphthoquinone methide with the protein under conditions effective to form the labeled protein.

2. The method of claim 1 wherein the protein comprises a cysteine residue having the thiol group.

3. The method of claim 1 wherein the o-naphthoquinone methide is a 2-naphthoquinone-3-methide.

4. The method of claim 1 wherein the o-naphthoquinone methide is of the formula: 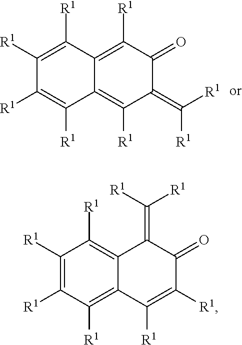
wherein each R¹ is independently H, halogen, or an organic group, wherein two or more R¹ groups may optionally be combined to form one or more rings.

5. The method of claim 1 wherein the labeling method is reversible.

6. The method of claim 1 further comprising:
irradiating the labeled protein under conditions effective to remove the label.

7. The method of claim 6 wherein conditions effective to remove the label comprise irradiating the labeled protein in a dilute solution.

8. The method of claim 6 wherein conditions effective to remove the label comprise irradiating the labeled protein in the presence of a polarized olefin.

9. The method of claim 8 wherein the polarized olefin is a vinyl ether.

10. The method of claim 1 wherein generating the o-naphthoquinone methide comprises:
providing an o-naphthoquinone methide precursor compound; and
irradiating the precursor compound under conditions effective to form the o-naphthoquinone methide.

11. The method of claim 10 wherein the precursor compound comprises a 3-hydroxymethyl-2-naphthol group.

12. The method of claim 10 wherein the precursor compound is of the formula: 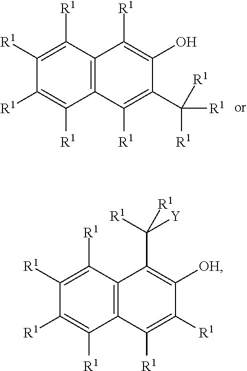
wherein:
each R¹ is independently H, halogen, or an organic group, wherein two or more R¹ groups may optionally be combined to form one or more rings;
Y is OR⁵, NR⁵₂, or NR⁵₃⁺ (Z_{1/q})⁻ wherein Z is an anion having a negative charge of q; and
each R⁵ is independently H or an organic group, wherein two or more R⁵ groups may optionally be combined to form one or more rings.

13. The method of claim 10 wherein conditions effective to form the labeled protein comprise irradiating a molar excess of the precursor compound compared to the moles of thiol groups of the protein.

14. The method of claim 10 wherein the precursor compound is irradiated in the presence of the protein.

15. A method for inhibiting an enzyme having a thiol group, the method comprising:
generating an o-naphthoquinone methide; and
contacting the o-naphthoquinone methide with the enzyme under conditions effective to react with the thiol group.

16. The method of claim 15 wherein the enzyme comprises a cysteine residue having the thiol group.

17. The method of claim 15 wherein the enzyme is a cysteine protease.

18. The method of claim 15 wherein the o-naphthoquinone methide is a 2-naphthoquinone-3-methide.

19. The method of claim 15 wherein the o-naphthoquinone methide is of the formula: 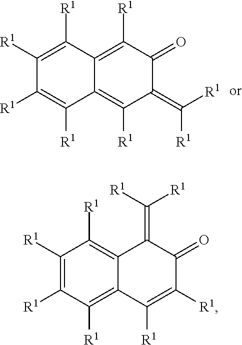
wherein each R¹ is independently H, halogen, or an organic group, wherein two or more R¹ groups may optionally be combined to form one or more rings.

20. The method of claim 15 wherein the inhibition method is reversible.

21. The method of claim 15 further comprising:
irradiating the inhibited enzyme under conditions effective to remove the reacted o-naphthoquinone methide and reform the thiol group.

22. The method of claim 21 wherein conditions effective to remove the reacted o-naphthoquinone methide fragment and reform the thiol group comprise irradiating the inhibited enzyme in a dilute solution.

23. The method of claim 21 wherein conditions effective to remove the reacted o-naphthoquinone methide fragment and reform the thiol group comprise irradiating the inhibited enzyme in the presence of a polarized olefin.

24. The method of claim 23 wherein the polarized olefin is a vinyl ether.

25. The method of claim 15 wherein generating the o-naphthoquinone methide comprises:
providing an o-naphthoquinone methide precursor compound; and
irradiating the precursor compound under conditions effective to form the o-naphthoquinone methide.

26. The method of claim 25 wherein the precursor compound comprises a 3-hydroxymethyl-2-naphthol group.

27. The method of claim 25 wherein the precursor compound is of the formula: 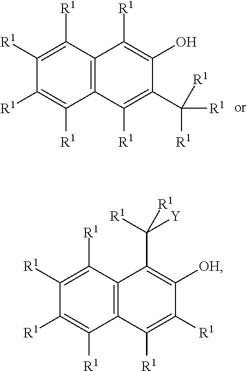
wherein:
each R¹ is independently H, halogen, or an organic group, wherein two or more R¹ groups may optionally be combined to form one or more rings;
Y is OR⁵, NR⁵₂, or NR⁵₃⁺(Z_{1/q})⁻ wherein Z is an anion having a negative charge of q; and
each R⁵ is independently H or an organic group, wherein two or more R⁵ groups may optionally be combined to form one or more rings.

28. The method of claim 25 wherein conditions effective to inhibit the enzyme comprise irradiating a molar excess of the precursor compound compared to the moles of the thiol group of the enzyme.

29. The method of claim 25 wherein the precursor compound is irradiated in the presence of the enzyme.
